(19) United States
(12) Patent Application Publication
(10) Pub. No.: US 2023/0099569 A1
(43) Pub. Date: Mar. 30, 2023

(54) INFLATION DEVICE CONNECTOR

(71) Applicant: Accumed Radial Systems, LLC, Farmington Hills, MI (US)

(72) Inventors: Joseph R. Korotko, Livonia, MI (US); Ronan, Winter Garden, FL (US)

(21) Appl. No.: 17/957,102

(22) Filed: Sep. 30, 2022

Related U.S. Application Data
(60) Provisional application No. 63/250,614, filed on Sep. 30, 2021.

(51) Int. Cl. A61M 25/10

(52) U.S. Cl. CPC A61M 25/1025; A61M 25/10186

(57) ABSTRACT

An example connector for a balloon catheter assembly according to an example of the present disclosure includes, among other possible things, an inlet branch, a first outlet branch in fluid communication with the inlet branch and configured to be connected to a first balloon, a second outlet branch in fluid communication with the inlet branch and configured to be connected to a second balloon, and a valve integrated the first outlet branch. The valve has a first position and a second position. The valve is configured to limit fluid flow through the valve when in the first position. Another example connector for a balloon catheter assembly and a method of inflating a balloon catheter assembly are also disclosed.

### CROSS-REFERENCE TO RELATED APPLICATIONS

This application claims priority U.S. Provisional Application No. 63/250,614 filed Sep. 30, 2021, which is hereby incorporated herein in its entirety.

### BACKGROUND

Some medical procedures utilize one or more balloon catheters. Certain procedures may benefit from the ability to easily inflate multiple balloon catheters.

### BRIEF DESCRIPTION OF THE DRAWINGS

FIG. 1 shows an example inflation device connector.

FIG. 2 shows another example inflation device connector.

FIG. 3 shows an exploded view of a valve for the example inflation device connector.

FIG. 4A shows an isometric view of the valve in an open position.

FIG. 4B shows a cross-sectional view of the valve in the open position.

FIG. 5A shows an isometric view of the valve in a one-way position.

FIG. 5B shows a cross-sectional view of the valve in the one-way position.

### SUMMARY

An example connector for a balloon catheter assembly according to an example of the present disclosure includes, among other possible things, an inlet branch, a first outlet branch in fluid communication with the inlet branch and configured to be connected to a first balloon, a second outlet branch in fluid communication with the inlet branch and configured to be connected to a second balloon, and a valve integrated the first outlet branch. The valve has a first position and a second position. The valve is configured to limit fluid flow through the valve when in the first position.

An example connector for a balloon catheter assembly according to an example of the present disclosure includes, among other possible things, a connector with an inlet branch, a first outlet branch in fluid communication with the inlet branch and configured to be connected to a first balloon, and a second outlet branch in fluid communication with the inlet branch and configured to be connected to a second balloon. The balloon catheter assembly also includes a valve in fluid communication with the first outlet branch. The valve has a first position and a second position, and the valve is configured to allow fluid flow through the valve when in the first position. The valve has a housing, a knob, and a one-way valve. A passageway is defined between first and second fluid ports, and the knob includes a receivable portion received in the housing and rotatable relative to the housing between the first position and the second position.

A method of inflating a balloon catheter assembly according to an example of the present invention includes, among other possible things, connecting a first balloon catheter to a first outlet branch of a connector, connecting a second balloon catheter to a second outlet branch of the connector, and opening a valve to inflate the first balloon catheter with fluid provided to an inlet branch of the connector. The inlet branch is in fluid communication with the first outlet branch of the connector. The valve integrated with the first outlet branch of the connector.

### DETAILED DESCRIPTION

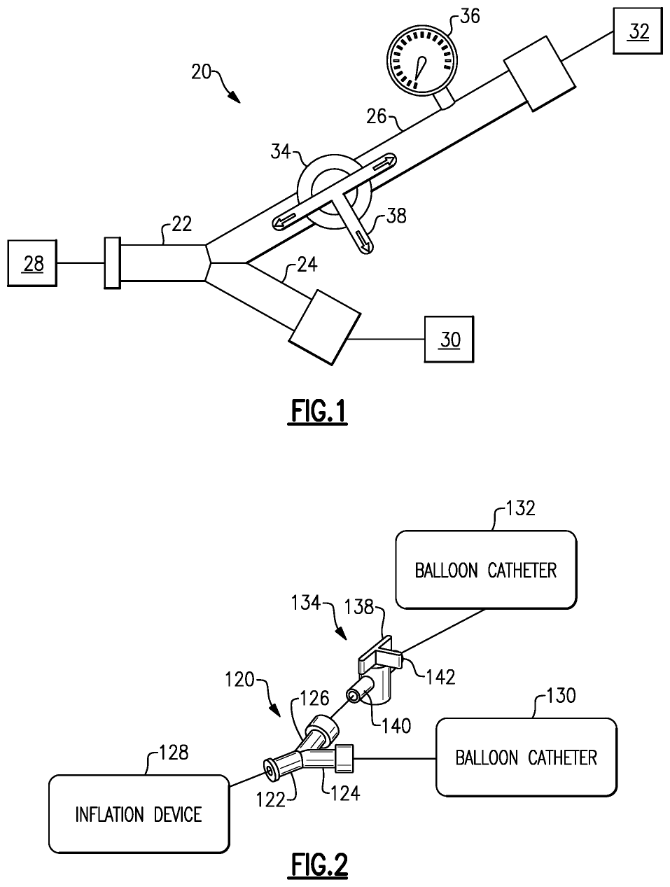

FIG. 1 shows an example connector 20 for use with a balloon catheter inflation device. The connector 20 is generally a y-shaped connector having an inlet branch 22, a first outlet branch 24, and a second outlet branch 26. The connector 20 connects an inflation device 28 to a first balloon 30 and a second balloon 32 (each shown schematically). The inflation device 28 may be any style of inflation device, such as a gun-style inflation device or a syringe style inflation device, for example. The balloons 30, 32 may be any type or size of balloon catheter. The balloons 30, 32 may be the same size as one another, or may be different sizes. A valve 34 is arranged along the second outlet branch 26 between the inflation device 28 and the second balloon 32. A pressure gauge 36 may also be arranged along the second outlet branch 26. The connector 20 is a single-piece integrated device. That is, the inlet branch 22, first outlet branch 24, and second outlet branch 26 are integral with one another and the valve 34 is integrated with the connector 20.

In one example the valve 34 is a valve that has an open position and a closed position. In the open position, fluid can flow in either direction through the valve 34, allowing the balloon 32 to be inflated or deflated. In the closed position, no fluid can flow through the valve 34. Another valve 34 could be included on the first outlet branch 24 between the inflation device 28 and the first balloon 30.

In another example, the valve 34 has at least two positions: an open position and a one-way position. The valve 34 has a knob 38 that allows a user to move the valve 34 between the open position and the one-way position. In the open position, fluid may flow in either direction into or out of the balloon 32. In the one-way position, fluid may flow out of the balloon 32 towards the inflation device 28 but may not flow into the balloon 32. That is, in the one-way position, the balloon 32 can be deflated but not inflated. Alternately, in the one-way position, the valve can allow for inflation only, and not deflation. The valve 34 can be switched between the open position and the one-way position as needed via the knob 38.

The connector 20 and valve 34 allows a user to inflate and deflate both balloons 30, 32 simultaneously. The connector 20 may be used in a heart procedure, for example. A physician or user could access the vascular arteries of a patient through an access point, which may be the radial artery at the patient's wrist or the femoral artery at the patient's groin, for example. A guide catheter and guidewire may be inserted into the patient from the access point. Two balloon catheters 30, 32 may then be inserted. The connector 20 connects both of the balloon catheters 30, 32 to a single inflation device 28. The physician or user can then inflate both balloons 30, 32 at the same time by having the valve 34 in the open position. Once the second balloon 32, which is on the branch 26 with the valve 34, is inflated to the desired pressure, the physician or user turns the valve 34 to the one-way position. The first balloon 30 can then be inflated to a greater pressure, if desired. Since the valve 34 is in the one-way position, fluid will not continue to travel into the second balloon 32. Once the physician or user is ready to deflate the balloons 30, 32, both the first and second balloons 30, 32 are deflated simultaneously. In one example, the inflation device 28 includes a syringe, and pulling the plunger creates a negative pressure so fluid travels out of the balloons 30, 32. Because the valve 34 is in the one-way position, fluid will travel out of the second balloon 32 without having to move the valve 34 to the open position.

The illustrated example shows a connector 20 having a single valve 34 and pressure gauge 36. In this example, the second balloon 32 which is on the outlet branch 26 with the valve 34 will be inflated to a pressure that is the same or lower than the pressure of the first balloon 30. In this example, the single pressure gauge 36 will display the pressure of the second balloon 32 and the physician or user could use a pressure gauge on the inflation device 28 to determine the pressure of the first balloon 30. In other examples, the connector 20 could have a valve 34 and/or a pressure gauge 36 on each outlet branch 24, 26. Although an angioplasty procedure is described, the example connector 20, 120 may be used for other medical procedures in which balloon catheters are used.

FIG. 2 shows another example inflation device connector. In this example, the connector 120 and valve 134 are separate components. To the extent not otherwise described or shown, the connector 120 corresponds to the connector 20 of FIG. 1, with like parts having reference numerals preappended with a “1.” In this example, a standard y-connector having an inlet branch 122 and two outlet branches 124, 126 is used, with the valve 134 being connected to one of the outlet branches 126. The valve 134 is connected to other medical devices such as the connector 120 and the second balloon 132 via connectors 140, 142. The connectors 140, 142 may be male or female luer connectors, for example. In one example, the valve 134 has a male luer connector 140 on one side and a female luer connector 142 on the other side. The further details of the valve 134 described herein may apply to a valve that is integral with a connector or a separate component from the connector.

In one example the valve 134 is a valve that has an open position and a closed position. In the open position, fluid can flow in either direction through the valve 134, allowing the balloon 132 to be inflated or deflated. In the closed position, no fluid can flow through the valve 134. Another valve 134 could be included on the first outlet branch 24 between the inflation device 128 and the first balloon 130.

In one example, the valve 20, 120 and the inflation device 28,128 are integrated together and into a single device, which may also include an integral pressure gauge 36.

FIG. 3 shows an exploded view of another example of the valve 134. The valve 134 includes a housing 144, a knob 138, and a one-way valve 146. The housing 144 includes the connectors 140, 142. A passageway 150 is defined through the housing between the connectors 140, 142. The knob 138 includes a bottom portion 148 that fits within the housing 144 and turns relative to the housing 144. The knob 138 has a one-way passageway 152 and an open passageway 154. The one-way valve 146 fits within the bottom portion 148 of the knob 138 and is arranged along the one-way passageway 152. The one-way passageway 152 and the open passageway 154 may be arranged at a right angle relative to one another, in one example.

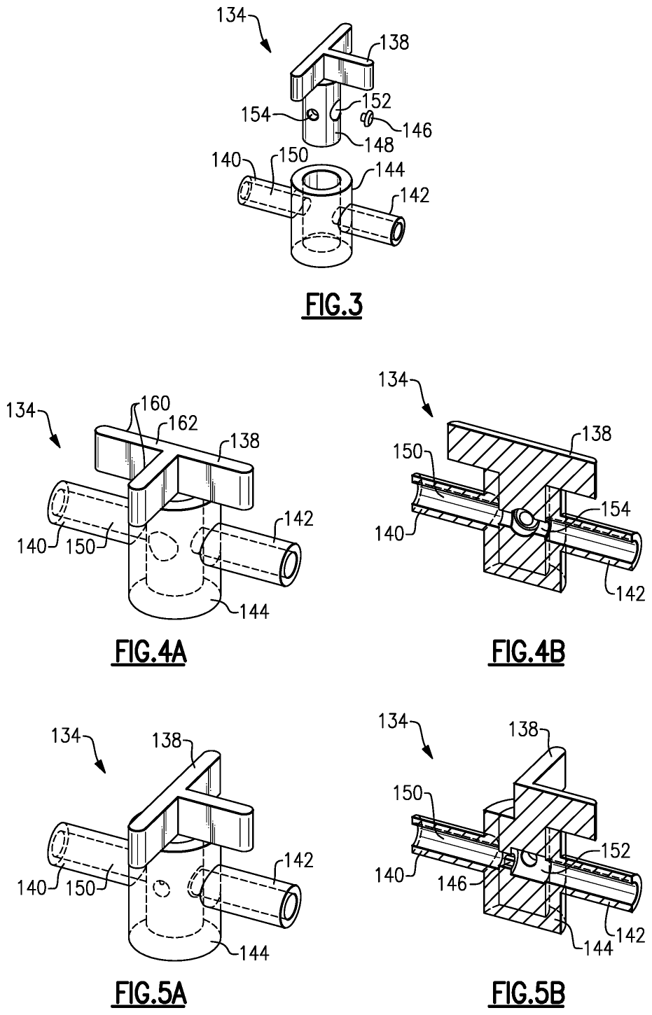

FIGS. 4A and 4B illustrate the valve 134 in the open position. In the illustrated example, the knob 138 has a plurality of protrusions 160 that indicate the open and one-way positions. The knob 138 may also include markings 162 such as arrows or words to indicate the valve position. This arrangement provides a visual indication to the physician or user whether the valve 134 is in the open or one-way position. Although a particular example knob 138 is illustrated, other knob arrangements may be used.

In the open position, the open passageway 154 is aligned with the passageway 150, such that fluid can flow in either direction between the connector 140 and the connector 142. In this position, the one-way passageway 152 and one-way valve 146 are blocked by the housing 144. In this open position, the second balloon 132 can be either inflated or deflated.

FIGS. 5A and 5B illustrate the valve 134 in the one-way position. In the one-way position, the one-way passageway 152 is aligned with the passageway 150. The one-way valve 146 is arranged within the one-way passageway 152 blocking the flow of fluid. In this position, fluid can flow through the passageways 150, 152 from the connector 140 to the connector 142, but cannot flow the opposite direction from the connector 142 to the connector 140. That is, the balloon 132 may be deflated but may not be inflated. This allows a physician or user to deflate both balloons 130, 132 simultaneously without having to open the valve 134. Although a one-way arrangement that allows deflation but not inflation is described, an arrangement that permits inflation but not deflation may be used in some situations. Although a particular valve design is shown, other one-way valve arrangements may be used. Common examples of one-way valve designs may be an ‘umbrella’ style one-way valve or a ‘duckbill’ style one-way valve, for example.

In other examples, the valve 134 is integrated with the connector 120 as in the example of FIG. 1 discussed above. In this example, the connectors 140, 142 may be omitted, but fluid ports to the valve 134 remain in their place.

In some prior systems, a valve needs to be moved to an open position in order to deflate the second balloon. However, in such systems, fluid can flow from the first balloon to the second balloon once the valve is opened before the user has the chance to start deflating the balloons when the first balloon is at a higher pressure than the second balloon. This may cause the second balloon to be inflated to a higher pressure than desired. The disclosed connector and valve arrangement permits the physician or user to deflate both balloons simultaneously without having to adjust the valve 34, 134, and without risk of fluid flowing from the first balloon to the second balloon before deflation. That is, the valve 34, 134 protects the lower pressure balloon from being exposed or pressurized to the higher pressure of the other balloon.

Further, although the valve 134 is described in combination with a y-connector, the valve 134 may be used without a y-connector for a single balloon. In this example, the valve 134 is attached to the inflation device via the connector 140 and attached to the balloon 132 via the connector 142. The valve 134 permits the physician or user to inflate the balloon 132 to the desired pressure with the valve 134 in the open position. The physician or user then turns the valve 134 to the one-way position. In the one-way position, the user can no longer inflate the balloon 132. The balloon 132 can then be deflated whenever needed without needing to reopen the valve 134.

Although the different examples have the specific components shown in the illustrations, embodiments of this disclosure are not limited to those particular combinations. It is possible to use some of the components or features from one of the examples in combination with features or components from another one of the examples.

Although an embodiment of this disclosure has been explained, a worker of ordinary skill in this art would recognize that certain modifications would come within the spirit and scope of this invention.

What is claimed is:

1. A connector for a balloon catheter assembly, comprising:
an inlet branch;
a first outlet branch in fluid communication with the inlet branch and configured to be connected to a first balloon;
a second outlet branch in fluid communication with the inlet branch and configured to be connected to a second balloon;
a valve integrated into the first outlet branch, the valve having a first position and a second position, wherein the valve is configured to limit fluid flow through the valve when in the first position.

2. The connector of claim 1, further comprising a valve integrated with the second outlet branch.

3. The connector of claim 1, further comprising a pressure gauge along the first outlet branch.

4. The connector of claim 1, wherein the first position precludes fluid flow through the valve.

5. The connector of claim 1, wherein the first position is configured to permit fluid flow in one direction through the valve and preclude fluid flow through a second direction opposite from the first direction through the valve.

6. The connector of claim 5, wherein the valve includes a housing, a knob, and a one-way valve, wherein a passageway is defined between first and second fluid ports, and wherein the knob includes a receivable portion received in the housing and rotatable relative to the housing between the first position and the second position.

7. The connector of claim 6, wherein the knob includes a one-way passageway and an open passageway, and wherein a one-way valve is received in the receivable portion of the knob and in the one-way passageway.

8. The connector of claim 7, wherein the one-way passageway and the open passageway are arranged at a right angle relative to one another.

9. A connector assembly for a balloon catheter, comprising:
a connector having an inlet branch, a first outlet branch in fluid communication with the inlet branch and configured to be connected to a first balloon, and a second outlet branch in fluid communication with the inlet branch and configured to be connected to a second balloon; and
a valve in fluid communication with the first outlet branch, the valve having a first position and a second position, wherein the valve is configured to allow fluid flow through the valve when in the first position, the valve including a housing, a knob, and a one-way valve, wherein a passageway is defined between first and second fluid ports, and wherein the knob includes a receivable portion received in the housing and rotatable relative to the housing between the first position and the second position.

10. The connector assembly of claim 9, wherein the knob includes a one-way passageway and an open passageway, and wherein a one-way valve is received in the receivable portion of the knob and in the one-way passageway.

11. The connector assembly of claim 10, wherein the one-way passageway and the open passageway are arranged at a right angle relative to one another.

12. The connector assembly of claim 9, wherein the valve is integrated with the first outlet branch.

13. The connector assembly of claim 9, wherein the valve is separate from the first outlet branch.

14. The connector assembly of claim 9, further comprising a valve integrated with the second outlet branch.

15. The connector assembly of claim 9, further comprising a pressure gauge along the first outlet branch.

16. A method of inflating a balloon catheter assembly, comprising:
connecting a first balloon catheter to a first outlet branch of a connector;
connecting a second balloon catheter to a second outlet branch of the connector; and
opening a valve to inflate the first balloon catheter with fluid provided to an inlet branch of the connector, the inlet branch in fluid communication with the first outlet branch of the connector, the valve integrated with the first outlet branch of the connector.

17. The method of claim 16, further comprising opening a valve to inflate the second balloon catheter with the fluid, the valve integrated with the second outlet branch of the connector.

18. The method of claim 16, further comprising closing the valve, thereby precluding fluid flow through the first outlet branch of the connector.

19. The method of claim 16, further comprising closing the valve, thereby allowing the first balloon catheter to be deflated but not inflated.

20. The method of claim 16, wherein the valve includes a housing, a knob, and a one-way valve, wherein a passageway is defined between first and second fluid ports, and wherein the knob includes a receivable portion received in the housing and rotatable relative to the housing, and wherein opening the valve includes rotating the knob relative to the housing.
